(19) United States
(12) Patent Application Publication
(10) Pub. No.: US 2017/0209355 A1
(43) Pub. Date: Jul. 27, 2017

(54) COMPOSITION

(71) Applicant: L'OREAL, Paris (FR)

(72) Inventors: Yoji TEZUKA, Kawasaki-shi, Kanagawa (JP); Kazuhiko MARUYAMA, Kawasaki-shi, Kanagawa (JP)

(21) Appl. No.: 15/514,593

(22) PCT Filed: Aug. 28, 2015
(86) PCT No.: PCT/JP2015/075143

§ 371 Date: Mar. 27, 2017

(30) Foreign Application Priority Data
Sep. 29, 2014 (JP) 2014-198229

(51) Int. Cl. A61K 8/44; A61K 8/39; A61Q 1/14; A61K 8/92; A61Q 19/10; A61Q 5/02; A61K 8/34; A61K 8/37

(52) U.S. Cl. CPC A61K 8/442; A61K 8/342; A61K 8/39; A61K 8/37; A61K 8/92; A61Q 19/10; A61Q 5/02; A61Q 1/14

(57) ABSTRACT

The present invention relates to a composition comprising: (a) at least one liquid oil at 25° C., (b) at least one wax, (c) at least one oil gelling agent derived from glutamic acid, and (d) at least one nonionic surfactant. The composition according to the present invention provides high removability and good feeling after rinsing-off without requiring further cleansing. In addition, the composition according to the present invention can provide high stability and good spread ability.

### TECHNICAL FIELD

The present invention relates to a composition which is suitable for cosmetic uses. Particularly, the present invention relates to a cosmetic composition for cleansing skin, nails, hair, eyelashes, eyebrows, eyes or mucous membranes.

### BACKGROUND ART

Currently, consumers use long-lasting makeup products such as transfer-resistant foundations, long-lasting lipsticks and waterproof mascaras. However, these types of makeup products are more difficult to remove than standard makeup products.

There are several types of makeup remover compositions such as milk, gel, cream and oil. Among these makeup remover compositions, oil type compositions are usually used due to their good removability. However, oil type compositions can easily drip down due to their low viscosity and are difficult to apply to skin. In addition, oil type compositions may provide greasy feeling after rinsing-off, and further cleansing may be required.

Therefore, solid type compositions have been proposed for removing makeup products. For example, JP-A-2005-298387 discloses a transparent solid cleansing agent comprising an amino acid derivative, a liquid oil and a nonionic surfactant. However, further cleansing is necessary in order to obtain non-greasy feeling after rinsing-off. Furthermore, more stability (e.g., less or no phase separation and the like) of the compositions over some factors such as temperature changes has been required.

Accordingly, there is still need for a stable composition for cleansing skin, nails, hair, eyelashes, eyebrows, eyes and/or mucous membranes, which can provide a non-greasy feeling after rinsing-off and prevent the composition from dripping down.

### DISCLOSURE OF INVENTION

An objective of the present invention is to provide a stable composition which is suitable for a cosmetic use such as cleansing skin, nails, hair, eyelashes, eyebrows, eyes and/or mucous membranes, which can provide a good feeling after rinsing-off without requiring further cleansing and can prevent the composition from dripping down.

The above objective can be achieved by a composition comprising:
(a) at least one liquid oil at 25° C.,
(b) at least one wax,
(c) at least one oil gelling agent derived from glutamic acid, and
(d) at least one nonionic surfactant.

It is preferable that the (b) wax(es) is a synthetic wax. The wax preferably comprises hydrocarbons containing odd and even numbers of carbon atoms, preferably Fisher-Tropsch wax.

The composition according to the present invention may further comprises (e) at least one fatty alcohol.

The amount of the (e) fatty alcohol(s) may be from 0.5 to 15% by weight, preferably from 1 to 10% by weight, and more preferably from 2 to 8% by weight, relative to the total weight of the composition.

The amount of the (a) liquid oil(s) at 25° C. may be from 50 to 90% by weight, preferably from 60 to 85% by weight, and more preferably from 65 to 80% by weight, relative to the total weight of the composition.

The amount of the (b) wax(es) may be from 1 to 15% by weight, preferably from 3 to 10% by weight, and more preferably 4 to 6% by weight, relative to the total weight of the composition.

It is preferable that the (c) oil gelling agent(s) is N-acyl glutamic acid dialkylamide. The N-acyl glutamic acid dialkylamide is preferably N-acyl glutamic acid dibutylamide.

The amount of the (c) oil gelling agent(s) may be from 0.01 to 5% by weight, preferably from 0.02 to 1% by weight, and more preferably from 0.03 to 0.5% by weight, relative to the total weight of the composition.

It is preferable that the HLB of the (d) nonionic surfactant(s) may be from 8 to 13, preferably 9 to 12, and more preferably 10 to 11.

The amount of the (d) nonionic surfactant(s) may be from 1 to 25% by weight, preferably from 5 to 20% by weight, and more preferably 8 to 17% by weight, relative to the total weight of the composition.

The composition according to the present invention is preferably solid at 25° C.

The composition according to the present invention may be for a cosmetic use, particularly for cleansing skin, nails, hair, eyelashes, eyebrows, eyes or mucous membranes.

### BEST MODE FOR CARRYING OUT THE INVENTION

The inventors performed diligent research and found that a composition including a specific combination of a liquid oil, a wax, a specific oil gelling agent and a nonionic surfactant could have good cleansing ability and stability, provide a good feeling after rinsing-off without further cleansing and prevent the composition from dripping down.

In addition, the composition according to the present invention can maintain the appearance thereof against temperature changes and the like, because it has high stability. Furthermore, the composition according to the present invention can have good spread ability.

Thus, the composition according to the present invention comprises:
(a) at least one liquid oil at 25° C.,
(b) at least one wax,
(c) at least one oil gelling agent derived from glutamic acid, and
(d) at least one nonionic surfactant.

Hereinafter, the composition according to the present invention will be explained in a more detailed manner.

(a) Liquid Oil at 25° C.

The composition comprises at least one liquid oil at 25° C. Two or more liquid oils may be used in combination.

The term “oil” means a fatty substance that is liquid at room temperature (25° C.) and at atmospheric pressure. The oil(s) may be volatile or non-volatile, preferably non-volatile.

The volatile or nonvolatile oils can be hydrocarbon oils, in particular of animal or vegetable origin, synthetic oils, silicone oils, fluorinated oils or their mixtures. Within the meaning of the present invention, the term “silicone oil” is understood to mean an oil comprising at least one silicon atom, and in particular at least one Si—O group. The term “hydrocarbon oil” is understood to mean an oil mainly comprising hydrogen and carbon atoms and optionally oxygen, nitrogen, sulfur and/or phosphorus atoms.

Nonvolatile Oils

Within the meaning of the present invention, the term “nonvolatile oil” is understood to mean an oil having a vapor pressure of less than 0.13 Pa (0.01 mmHg). The nonvolatile oils can be chosen in particular from nonvolatile hydrocarbon oils, if appropriate fluorinated, and/or nonvolatile silicone oils.

Mention may in particular be made, as nonvolatile hydrocarbon oil suitable for use in the invention, of:
hydrocarbon oils of animal origin,
hydrocarbon oils of vegetable origin, such as phytosteryl esters, such as phytosteryl oleate, phytosteryl isostearate and lauroyl/octyldodecyl/phytosteryl glutamate, for example sold under the name Eldew PS203 by Ajinomoto, triglycerides composed of fatty acid esters of glycerol, the fatty acids of which can have varied chain lengths from C₄ to C₂₄, it being possible for the latter to be linear or branched and saturated or unsaturated; these oils are in particular heptanoic or octanoic triglycerides, wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkinseed oil, cucumber oil, blackcurrant oil, evening primrose oil, millet oil, barley oil, quinoa oil, rye oil, safflower oil, candlenut oil, passionflower oil or musk rose oil; shea butter; or alternatively caprylic/capric acid triglycerides, such as those sold by Stearineries Dubois or those sold under the names Miglyol 810®, 812® and 818® by Dynamit Nobel; or the refined vegetable perhydrosqualene sold under the name Fitoderm by Cognis;
hydrocarbon oils of mineral or synthetic origin, such as, for example:
   synthetic ethers having from 10 to 40 carbon atoms,
   linear or branched hydrocarbons of mineral or synthetic origin, such as liquid petroleum, polydecenes, hydrogenated polyisobutene, such as Parleam, squalane and their mixtures, in particular hydrogenated polyisobutene,
   synthetic esters, such as oils of formula R₁COOR₂ in which R₁ represents the residue of a linear or branched fatty acid comprising from 1 to 40 carbon atoms and R₂ represents a hydrocarbon chain, in particular a branched hydrocabon chain, comprising from 1 to 40 carbon atoms, provided that R₁+R₂ is ≧10,
      the esters can in particular be chosen from esters, in particular fatty acid esters, such as, for example:
      dicaprylyl carbonate (Cetiol CC from Cognis), cetearyl octanoate, esters of isopropyl alcohol, such as isopropyl myristate, isopropyl palmitate, ethyl palmitate, 2-ethylhexyl palmitate, isopropyl stearate, isopropyl isostearate, isostearyl isostearate, octyl stearate, hydroxylated esters, such as isostearyl lactate, octyl hydroxystearate, diisopropyl adipate, heptanoates, in particular isostearyl heptanoate, octanoates, decanoates or ricinoleates of alcohols or polyalcohols, such as propylene glycol dioctanoate, cetyl octanoate, tridecyl octanoate, 2-ethylhexyl 4-diheptanoate, 2-ethylhexyl palmitate, alkyl benzoate, polyethylene glycol diheptanoate, propylene glycol di(2-ethylhexanoate) and their mixtures, benzoates of C₁₂ to C₁₅ alcohols, hexyl laurate, neopentanoic acid esters, such as isodecyl neopentanoate, isotridecyl neopentanoate, isostearyl neopentanoate or octyldodecyl neopentanoate, isononanoic acid esters, such as isononyl isononanoate, isotridecyl isononanoate or octyl isononanoate, or hydroxylated esters, such as isostearyl lactate or diisostearyl malate,
      polyol esters and pentaerythritol esters, such as dipentaerythrityl tetrahydroxystearate/tetraisostearate,
      esters of dimer diols and of dimer diacids, such as Lusplan DD-DA5® and Lusplan DD-DA7®, sold by Nippon Fine Chemical and described in patent application FR 03 02809,
   fatty alcohols which are liquid at ambient temperature, comprising a branched and/or unsaturated carbon chain having from 12 to 26 carbon atoms, such as 2-octyldodecanol, isostearyl alcohol, oleyl alcohol, 2-hexyldecanol, 2-butyloctanol and 2-undecylpentadecanol,
   higher fatty acids, such as oleic acid, linoleic acid, linolenic acid and their mixtures, and
   dialkyl carbonates, it being possible for the two alkyl chains to be identical or different, such as dicaprylyl carbonate, sold under the name Cetiol CC® by Cognis,
   nonvolatile silicone oils, such as, for example, nonvolatile polydimethylsiloxanes (PDMSs), polydimethylsiloxanes comprising alkyl or alkoxy groups which are pendent and/or at the ends of the silicone chain, which groups each have from 2 to 24 carbon atoms, phenyl silicones, such as phenyl trimethicones, phenyl dimethicones, phenyl(trimethylsiloxy)diphenylsiloxanes, diphenyl dimethicones, diphenyl(methyldiphenyl)trisiloxanes and (2-phenylethyl)trimethylsiloxysilicates, dimethicones or phenyl trimethicones with a viscosity of less than or equal to 100 cSt, and their mixtures;
and their mixtures.

Volatile Oils

Within the meaning of the present invention, the term “volatile oil” is understood to mean an oil (or nonaqueous medium) which is capable of evaporating on contact with the skin in less than one hour, at ambient temperature and at atmospheric pressure. The volatile oil is a volatile cosmetic oil which is liquid at ambient temperature, having in particular a nonzero vapor pressure at ambient temperature and atmospheric pressure, especially having a vapor pressure ranging from 0.13 Pa to 40 000 Pa (10−3 to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile hydrocarbon oils can be chosen from hydrocarbon oils having from 8 to 16 carbon atoms, in particular branched C₈-C₁₆ alkanes (also known as isoparaffins), such as isododecane (also known as 2,2,4,4,6-pentamethylheptane), isodecane or isohexadecane, for example the oils sold under the Isopar® or Permethyl® trade names.

Use may also be made, as volatile oils, of volatile silicones, such as, for example, volatile linear or cyclic silicone oils, in particular those having a viscosity ≦8 centistokes (8×10⁻⁶ m²/s), and having in particular from 2 to 10 silicon atoms and especially from 2 to 7 silicon atoms, these silicones optionally comprising alkyl or alkoxy groups having from 1 to 10 carbon atoms.

Mention may in particular be made, as volatile silicone oil which can be used in the invention, of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane and their mixtures.

Use may also be made of volatile fluorinated oils, such as nonafluoromethoxybutane or perfluoromethylcyclopentane, and their mixtures.

It is also possible to use a mixture of the oils mentioned above.

The oils used in the composition according to the present invention are different from the (d) nonionic surfactants and (e) fatty alcohols described below.

The (a) oil may be a non-polar oil such as a hydrocarbon oil, a silicone oil, or the like; a polar oil such as a plant or animal oil and an ester oil or an ether oil; or a mixture thereof.

It is preferable that the (a) oil be selected from the group consisting of oils of plant or animal origin, synthetic oils, silicone oils and hydrocarbon oils.

The liquid oil according to the present invention is preferably an ester oil. More preferably, the liquid oil according to the present invention is ethylhexyl palmitate.

The amount of the liquid oil(s) at 25° C. in the composition according to the present invention may be from 50 to 90% by weight, preferably from 60 to 85% by weight, and more preferably from 65 to 80% by weight, relative to the total weight of the composition. If the amount of the liquid oil(s) is lower than 50% by weight, the removability of the composition would be worse. If the amount of the liquid oil(s) is higher than 90% by weight, the stability of the composition would be worse.

(b) Wax

The composition comprises at least one wax. Two or more waxes may be used in combination.

The term “wax” means a lipophilic compound, which is solid at room temperature (25° C.), with a reversible solid/liquid change of state, having a melting point of greater than or equal to 30° C., which may be up to 120° C. In particular, the waxes have a melting point of greater than or equal to 30° C. and better still greater than or equal to 45° C.

For the purposes of the invention, the melting point is measured, for example in accordance with ASTM D127.

The wax used in the composition according to the invention advantageously has a melting point of greater than or equal to 50° C., preferably greater than or equal to 60° C., and more preferably from 75 to 90° C.

The wax advantageously has a hardness at 20° C. of greater than 5 MPa, and especially ranging from 5 to 15 MPa.

The hardness of the wax is determined by measuring the compressive force, measured at 20° C. using the texturometer sold under the name TA-XT2 by the company Rheo, equipped with a stainless-steel cylinder 2 mm in diameter, travelling at a measuring speed of 0.1 mm/second, and penetrating the wax to a penetration depth of 0.3 mm.

The measuring protocol of the hardness is as follows:

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the hardness or the tack.

The texturometer spindle is displaced at a speed of 0.1 mm/s, and then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.5 mm/s.

The hardness value is the maximum compression force measured divided by the area of the texturometer cylinder in contact with the wax.

Examples of the wax used in the present invention include a natural wax and a synthetic wax. Examples of the natural wax include a petroleum wax, a plant wax, and an animal wax. Examples of the petroleum wax include a paraffin wax, a microcrystalline wax, and a petrolatum. Examples of the plant wax include rice wax, carnauba wax, candelilla wax, ouricury wax, Japan wax, cocoa butter, cork fibre wax and sugarcane wax. Examples of the animal wax include lanolin wax, lanolin derivatives and beeswax. Examples of the synthetic wax include a synthetic hydrocarbon wax and a modified wax.

Examples of the synthetic hydrocarbon wax include polyethylene wax, polypropylene wax, and Fischer-Tropsch wax. Examples of the modified wax include a paraffin wax derivative, a montan wax derivative, and a microcrystalline wax derivative. It is preferable that the (b) wax be selected from the synthetic hydrocarbon wax such as a polyethylene wax, a polypropylene wax, and a Fischer-Tropsch wax.

Examples of the polyethylene wax include an ethylene homopolymer and an ethylene-alpha-olefin copolymer. Alternatively, the wax may be obtained by thermal decomposition of the copolymer. Examples of the alpha-olefin include an alpha -olefin having 3 to 12 carbon atoms such as propylene, 1-butene, 1-pentene, 1-hexene, 4-methyl-l-pentene, and 1-octene.

Examples of the polypropylene wax include a propylene homopolymer, an ethylene-propylene copolymer (which is a random or block copolymer), propylene-alpha -olefin (except for ethylene or propylene) copolymer. Alternatively, the wax may be obtained by thermal decomposition of the copolymer. Examples of the alpha -olefin include 1-butene, 1-pentene, 1-hexene, 1-heptene, 1-octene, 4-methyl-1-pentene, 1-decene, 1-dodecene, 1-tetradecene, 1-hexadecene, and 1-octadecene.

The polyethylene wax and the polypropylene wax can be obtained by a known method using a polymerization catalyst such as a Ziegler catalyst, a Ziegler-Natta catalyst, and a metallocene catalyst. In particular, the polyethylene wax and the polypropylene wax obtained by using a metallocene catalyst as a polymerization catalyst are preferable, having a narrow molecular weight distribution and stable quality, in comparison with the polyethylene wax and the polypropylene wax obtained by using a Ziegler catalyst or a Ziegler-Natta catalyst as a polymerization catalyst.

The Fischer-Tropsch wax is a synthetic hydrocarbon wax mainly comprising linear hydrocarbons, which is obtained by reacting water gas containing carbon monoxide and hydrogen as main components under normal pressure at 170 to 250° C. using a catalyst such as cobalt, nickel, or iron. The Fischer-Tropsch wax is characterized in comprising hydrocarbons containing odd and even numbers of carbon atoms, namely comprising both hydrocarbons containing odd numbers of carbon atoms and hydrocarbons containing even numbers of carbon atoms. Most preferably, the wax according to the present invention is Fisher-Tropsch wax.

The amount of the wax(es) in the composition according to the present invention may be from 1 to 15% by weight, preferably from 3 to 10% by weight, and more preferably from 4 to 6% by weight, relative to the total weight of the composition. If the amount of the wax(es) is lower than 1% by weight, the stability of the composition would be worse. If the amount of the wax(es) is higher than 15% by weight, the spread ability of the composition would be worse.

(c) Oil Gelling Agent Derived from Glutamic Acid

The composition comprises at least one oil gelling agent derived from glutamic acid. Two or more oil gelling agents may be used in combination.

The oil gelling agents are capable of establishing, between themselves, physical interactions leading to self-aggregation of the molecules with formation of a 3D macromolecular network which is responsible for the gelation of the compound.

The macromolecular network may result from the formation of a network of fibrils (owing to stacking or aggregation of oil gelling molecules), which immobilizes the molecules in the compound. The ability to form this network of fibrils, and hence to gel, depends on the nature (or chemical class) of the oil gelling agent, on the nature of the substituents carried by its molecules for a given chemical class, and on the nature of the composition.

In one embodiment, the oil gelling agent according to the present invention is N-acyl glutamic acid derivative. N-acyl glutamic acid derivatives include N-acyl glutamic acid amides and N-acyl glutamic acid esters. Particularly preferred are N-acyl glutamic acid amides and N-acyl glutamic acid esters corresponding to the following formula:

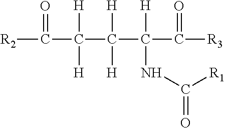

wherein R₁ is an alkyl, aryl, arylalkyl radical (branched, linear or cyclic), having from 1 to 26 carbon atoms; preferably, from 8 to 22 carbon atoms; more preferably, from 12 to 18 carbon atoms. R₂ and R₃ are the same or different, preferably the same, alkyl, aryl, arylalkyl ester radical or amide radical, in which the alkyl, aryl, arylalkyl moiety (branched, linear or cyclic) has from 1 to 26 carbon atoms; preferably, from 2 to 20 carbon atoms.

Examples of N-acyl glutamic acid derivatives that may be mentioned include N-lauroyl-glutamic acid diethyl amide, N-lauroyl-glutamic acid dibutyl amide, N-lauroyl-glutamic acid dihexyl amide, N-lauroyl-glutamic acid dioctyl amide, N-lauroyl-glutamic acid didecyl amide, N-lauroyl-glutamic acid didodecyl amide, N-lauroyl-glutamic acid ditetradecyl amide, N-lauroyl-glutamic acid dihexadecyl amide, N-lauroyl-glutamic acid distearyl amide, N-stearoyl-glutamic acid dibutyl amide, N-stearoyl-glutamic acid dihexyl amide, N-stearoyl-glutamic acid diheptyl amide, N-stearoyl-glutamic acid dioctyl amide, N-stearoyl-glutamic acid didecyl amide, N-stearoyl-glutamic acid didodecyl amide, N-stearoyl-glutamic acid ditetradecyl amide, N-stearoyl-glutamic acid dihexadecyl amide, N-stearoyl-glutamic acid distearyl amide and mixtures thereof, more preferred, is N-lauroyl-glutamic acid dibutyl amide, N-stearyl-glutamic acid dihexyl amide, and mixtures thereof.

In one embodiment, N-acyl glutamic acid amides in which the acyl group represents a C₈ to C₂₂ alkyl chain are particularly preferred. The oil gelling agent is preferably N-acyl glutamic acid dialkylamide, and more preferably N-lauroyl-L-glutamic acid dibutylamide, manufactured or sold by Ajinomoto under the name GP-1.

The amount of the oil gelling agent(s) may be from 0.01 to 5% by weight, preferably from 0.02 to 1% by weight, and more preferably from 0.03 to 0.5% by weight, relative to the total weight of the composition. If the amount of the oil gelling agent(s) is lower than 0.01% by weight, the stability of the composition would be worse. If the amount of the oil gelling agent(s) is higher than 5% by weight, the spread ability of the composition would be worse.

(d) Nonionic Surfactant

The composition comprises at least one nonionic surfactant. Two or more nonionic surfactants may be used in combination.

The nonionic surfactants are compounds well known in themselves (see, e.g., in this regard, “Handbook of Surfactants” by M. R. Porter, Blackie & Son publishers (Glasgow and London), 1991, pp. 116-178). Thus, they can, for example, be chosen from alcohols, alpha-diols, alkylphenols and esters of fatty acids, these compounds being ethoxylated, propoxylated or glycerolated and having at least one fatty chain comprising, for example, from 8 to 30 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils of plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives; amine oxides such as (C₁₀-C₁₄)alkylamine oxides or N—(C₁₀-C₁₄)acylaminopropylmorpholine oxides; and mixtures thereof.

The nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

Examples of polyoxyalkylenated nonionic surfactants that may be mentioned include: polyoxyalkylenated (C₈-C₂₄)alkylphenols,

saturated or unsaturated, linear or branched, polyoxyalkylenated C₈-C₃₀ alcohols,

saturated or unsaturated, linear or branched, polyoxyalkylenated C₈-C₃₀ amides,

esters of saturated or unsaturated, linear or branched, C₈-C₃₀ acids and of polyalkylene glycols, polyoxyalkylenated esters of saturated or unsaturated, linear or branched, C₈-C₃₀ acids and of sorbitol,

saturated or unsaturated, polyoxyalkylenated plant oils,

condensates of ethylene oxide and/or of propylene oxide, inter alia, alone or as mixtures.

The surfactants preferably contain a number of moles of ethylene oxide and/or of propylene oxide of between 2 and 100 and most preferably between 2 and 50. Advantageously, the nonionic surfactants do not comprise any oxypropylene units.

In accordance with one preferred embodiment of the invention, the polyoxyalkylenated nonionic surfactants are chosen from polyoxyethylenated fatty alcohol (polyethylene glycol ether of fatty alcohol) and polyoxyethylenated fatty ester (polyethylene glycol ester of fatty acid).

Examples of polyoxyethylenated fatty alcohol (or C₈-C₃₀ alcohols) that may be mentioned include the adducts of ethylene oxide with lauryl alcohol, especially those containing from 9 to 50 oxyethylene units and more particularly those containing from 10 to 12 oxyethylene units (Laureth-10 to Laureth-12, as the CTFA names); the adducts of ethylene oxide with behenyl alcohol, especially those containing from 9 to 50 oxyethylene units (Beheneth-9 to Beheneth-50, as the CTFA names); the adducts of ethylene oxide with cetearyl alcohol (mixture of cetyl alcohol and stearyl alcohol), especially those containing from 10 to 30 oxyethylene units (Ceteareth-10 to Ceteareth-30, as the CTFA names); the adducts of ethylene oxide with cetyl alcohol, especially those containing from 10 to 30 oxyethylene units (Ceteth-10 to Ceteth-30, as the CTFA names); the adducts of ethylene oxide with stearyl alcohol, especially those containing from 10 to 30 oxyethylene units (Steareth-10 to Steareth-30, as the CTFA names); the adducts of ethylene oxide with isostearyl alcohol, especially those containing from 10 to 50 oxyethylene units (Isosteareth-10 to Isosteareth-50, as the CTFA names); and mixtures thereof.

As examples of polyglycerolated nonionic surfactants, polyglycerolated C₈-C₄₀ alcohols are preferably used.

In particular, the polyglycerolated C₈-C₄₀ alcohols correspond to the following formula:

RO—[CH₂—CH(CH₂OH)—O]ₘ—H or RO—[CH(CH₂OH)—CH₂O]ₘ—H

in which R represents a linear or branched C₈-C₄₀ and preferably C₈-C₃₀ alkyl or alkenyl radical, and m represents a number ranging from 1 to 30 and preferably from 1.5 to 10.

As examples of compounds that are suitable in the context of the invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

Examples of polyoxyethylenated fatty esters that may be mentioned include the adducts of ethylene oxide with esters of lauric acid, palmitic acid, stearic acid or behenic acid, and mixtures thereof, especially those containing from 9 to 100 oxyethylene units, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

According to one of the embodiments according to the present invention, the nonionic surfactant may be selected from esters of polyols with fatty acids with a saturated or unsaturated chain containing for example from 8 to 24 carbon atoms, preferably 12 to 22 carbon atoms, and polyoxyalkylenated derivatives thereof, preferably containing from 10 to 200, and more preferably from 10 to 100 oxyalkylene units, such as glyceryl esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; sorbitol esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; sugar (sucrose, glucose, alkylglycose) esters of a C₈-C₂₄, preferably C₁₂-C₂₂, fatty acid or acids and polyoxyalkylenated derivatives thereof, preferably containing from 10 to 200, and more preferably from 10 to 100 oxyalkylene units; ethers of fatty alcohols; ethers of sugar and a C₈-C₂₄, preferably C₁₂-C₂₂, fatty alcohol or alcohols; and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can be cited, and as polyoxyalkylenated derivatives thereof, mono-, di- or triester of fatty acids with a polyoxyalkylenated glycerol (mono-, di- or triester of fatty acids with a polyalkylene glycol ether of glycerol), preferably polyoxyethylenated glyceryl stearate (mono-, di- and/or tristearate), such as PEG-20 glyceryl stearate (mono-, di- and/or tristearate) can be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEGIN by Goldschmidt (CTFA name: glyceryl stearate SE), can also be used.

The sorbitol esters of C₈-C₂₄ fatty acids and polyoxyalkylenated derivatives thereof can be selected from sorbitan palmitate, sorbitan trioleate and esters of fatty acids and alkoxylated sorbitan containing for example from 20 to 100 EO, such as for example polyethylene sorbitan trioleate (polysorbate 85) or the compounds marketed under the trade names Tween 20 or Tween 60 by Ubiqema.

As esters of fatty acids and glucose or alkylglucose, glucose palmitate, alkylglucose sesquistearates such as methylglucose sesquistearate, alkylglucose palmitates such as methylglucose or ethylglucose palmitate, methylglucoside fatty esters, the diester of methylglucoside and oleic acid (CTFA name: Methyl glucose dioleate), the mixed ester of methylglucoside and the mixture of oleic acid/hydroxystearic acid (CTFA name: Methyl glucose dioleate/hydroxystearate), the ester of methylglucoside and isostearic acid (CTFA name: Methyl glucose isostearate), the ester of methylglucoside and lauric acid (CTFA name: Methyl glucose laurate), the mixture of monoester and diester of methylglucoside and isostearic acid (CTFA name: Methyl glucose sesqui-isostearate), the mixture of monoester and diester of methylglucoside and stearic acid (CTFA name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (CTFA name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (CTFA name: PEG-20 methyl glucose sesquistearate) and in particular the product marketed under the name Glucamate SSE-20 by AMERCHOL and that marketed under the name Grillocose PSE-20 by GOLDSCHMIDT, and mixtures thereof, can for example be cited.

As sucrose esters, saccharose palmito-stearate, saccharose stearate and saccharose monolaurate can for example be cited.

As sugar ethers, alkylpolyglucosides can be used, and for example decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and mixtures thereof can in particular be cited.

Mixtures of glycerides of alkoxylated plant oils such as mixtures of ethoxylated (200 EO) palm and copra (7 EO) glycerides can also be cited.

The nonionic surfactant according to the present invention preferably contains alkenyl or branched C₁₂-C₂₂ acyl chain such as oleyl or isostearyl group. More preferably, the nonionic surfactant according to the present invention is PEG-20 glyceryl triisostearate.

The HLB of the nonionic surfactant(s) is preferably from 8 to 13, more preferably 9 to 12, and even more preferably 10 to 11. If two or more nonionic surfactants are used, the HLB value is determined by the weight average of the HLB values of all the nonionic surfactants. The HLB is the ratio between the hydrophilic part and the lipophilic part in the molecule. This term HLB is well known to those skilled in the art and is described in “The HLB system. A time-saving guide to emulsifier selection” (published by ICI Americas Inc., 1984). If the HLB of the nonionic surfactant(s) is lower than 8, the oily feeling after rinsing-off would remain. If the HLB of the nonionic surfactant(s) is higher than 13, the removability of the composition would be worse.

The amount of the (d) nonionic surfactant(s) may be from 1 to 25% by weight, preferably from 5 to 20% by weight, and more preferably 8 to 17% by weight, relative to the total weight of the composition. If the amount of the nonionic surfactant(s) is lower than 1% by weight, the feeling after rinsing-off would be worse. If the amount of the nonionic surfactant(s) is higher than 25% by weight, the stability of the composition and the feeling after rinsing-off would be worse.

(e) Fatty Alcohol

The composition according to the present invention may comprise at least one fatty alcohol. Two or more fatty alcohols may be used in combination.

The term “fatty alcohol” means any saturated or unsaturated, linear or branched C₈-C₃₀ fatty alcohol, which is optionally substituted, in particular with one or more hydroxyl groups (in particular 1 to 4). If they are unsaturated, these compounds may comprise one to three conjugated or non-conjugated carbon-carbon double bonds.

Among the C₈-C₃₀ fatty alcohols, C₁₆-C₂₂ fatty alcohols, for example, are used. Mention may be made among these of cetyl alcohol, stearyl alcohol, isostearyl alcohol, oleyl alcohol, behenyl alcohol, linoleyl alcohol, palmitoleyl alcohol, linolenyl alcohol, arachidonyl alcohol, erucyl alcohol and octyldodecanol, and mixtures thereof. In one embodiment, octyldodecanol can be used as a fatty alcohol.

The amount of the fatty alcohol(s) may be from 0.5 to 15% by weight, preferably from 1 to 10% by weight, and more preferably from 2 to 8% by weight, relative to the total weight of the composition. If the amount of the fatty alcohol(s) is lower than 0.5% by weight, the stability and the spread ability of the composition would be worse. If the amount of the fatty alcohol(s) is higher than 15% by weight, the stability and the spread ability of the composition would be worse.

The compositions of the present invention may also comprise additives that are common in cosmetics, such as antioxidants, fragrances, fragrance peptizers, fillers, dyestuffs and hydrophilic or lipophilic active agents. The nature of the adjuvants and the amounts thereof should be such that they do not modify the properties of the composition according to the present invention. The amounts of these additives are those conventionally used in the cosmetics field, for example from 0.001% to 10% by weight, relative to the total weight of the composition.

As active agents that may be used in the composition of the present invention, examples that may be mentioned include calmatives such as allantoin and bisabolol; glycyrrhetinic acid and salts thereof; antibacterial agents such as octopirox, triclosan and triclocarban; essential oils; vitamins, for instance retinol (vitamin A), ascorbic acid (vitamin C), tocopherol (vitamin E), niacinamide (vitamin PP or B3), panthenol (vitamin B5) and derivatives thereof, for instance esters of these vitamins (palmitate, acetate, propionate), magnesium ascorbyl phosphate, glycosyl vitamin C or glucopyranosyl ascorbic acid (ascorbyl glucoside); coenzymes such as coenzyme Q10 or ubiquinone and coenzyme R or biotin; protein hydrolysates; plant extracts and especially plankton extracts; and mixtures thereof.

The composition according to the present invention is preferably solid at 25° C. In one embodiment, the composition according to the present invention preferably has a melting point or a thermal transition temperature such as the softening point of greater than 25° C., which may especially range from 25 to 85° C., or even from 30 to 60° C. and in particular from 30 to 45° C. and/or a hardness that may range from 0.001 to 0.5 MPa and especially from 0.005 to 0.4 MPa.

In one embodiment, the composition according to the present invention can have the appearance of a cream, an ointment, a soft paste, a salve, or a cast or molded solid, such as a stick.

The composition according to the present invention can be prepared by mixing the above essential or optional components by using a conventional mixing means such as a mixer and a homogenizer. Preferably, (a) at least one liquid oil, (b) at least one wax and optionally other ingredients are mixed and heated preferably to 70-100° C. to prepare an oil phase, and (c) at least one oil gelling agent derived from glutamic acid, (d) at least one nonionic surfactant, and optionally other ingredients including (d) at least one fatty alcohol are mixed and heated preferably to 80-110° C. to prepare a surfactant phase, and then the oil phase and the surfactant are mixed well preferably at 70-100° C.

The compositions of the present invention can be used in any cosmetic application. The compositions according to the invention are preferably in the form of balm, and they can in particular constitute cosmetic or dermatological compositions, for example cosmetic compositions for the treatment of keratin materials such as the skin, the mucous membranes, the eyelashes, the hair and the nails. They can also constitute, for example, makeup-removing compositions and/or cleansing compositions and/or care compositions for the skin, the mucous membranes such as the lips, and/or for the eyelashes, compositions for massaging facial skin or body skin, scrubbing (or exfoliating) compositions both for the face and for the hands (when the composition contains exfoliating particles). The compositions according to the invention can also be used as shower care balms.

Preferably, the composition according to the present invention is for cleansing skin, nails, hair, eyelashes, eyebrows, eyes or mucous membranes. The composition is particularly suited to remove makeup from the skin and/or the area around the eyes and/or from the lips.

### EXAMPLES

The present invention will be described in more detail by way of examples, which however should not be construed as limiting the scope of the present invention.

### Examples 1 to 6 and Comparative Examples 1 to 4

The following compositions according to Examples 1 to 6 and Comparative Examples 1 to 4, shown in Table 1, were prepared. Ethylhexyl palmitate, waxes (Fischer Tropsch wax, polyethylene or microcrystalline wax), Butyrospermum Parkii (Shea) butter, hydrogenated castor oil dimer dilinoleate and phenoxyethanol were mixed and heated to around 90° C. to prepare an oil phase. Then, PEG-20 glyceryltriisostearate, octyldodecanol, dibutyl lauroyl glutamide and caprylyl glycol were mixed and heated to around 95° C. to prepare a surfactant phase. The oil phase and the surfactant phase were then mixed well at around 90° C. Tocopherol and fragrance were then mixed into the mixture and the mixture was cooled to room temperature.

The numerical values for the amounts of the components shown in the Table 1 are all based on “% by weight”.

TABLE 1

| | | | | | | | Comparative | | | |
|---|---|---|---|---|---|---|---|---|---|---|
| | Examples | | | | | | Examples | | | |
| Name | 1 | 2 | 3 | 4 | 5 | 6 | 1 | 2 | 3 | 4 |
| (a) Ethylhexyl Palmitate | 69.7 | 73.7 | 73.4 | 77.2 | 69.7 | 69.7 | 77.5 | 70.0 | 83.7 | 74.7 |
| (b) Fischer-Tropsch wax | 5.0 | 5.0 | 4.0 | 5.0 | — | — | 5.0 | 5.0 | 5.0 | — |
| (mp. 79-84° C.) | | | | | | | | | | |
| (b) Polyethylene wax | — | — | — | — | 5.0 | — | — | — | — | — |
| (mp. 83-91° C.) | | | | | | | | | | |
| (b) Microcrystalline | — | — | — | — | — | 5.0 | — | — | — | — |
| wax (mp. 82-92° C.) | | | | | | | | | | |
| (c) N-lauroyl-L-glutamic | 0.3 | 0.3 | 0.05 | 0.3 | 0.3 | 0.3 | — | — | 0.3 | 0.3 |
| acid dibutylamide | | | | | | | | | | |
| (d) PEG-20 Glyceryl | 14 | 10 | 14 | 14 | 14 | 14 | 14 | 14 | — | 14 |
| triisostearate | | | | | | | | | | |
| (HLB = 10.2) | | | | | | | | | | |
| (e) Oetyldodecanol | 7.5 | 7.5 | 3.0 | — | 7.5 | 7.5 | — | 7.5 | 7.5 | 7.5 |
| Hydrogenated castor | — | — | 2.0 | — | — | — | — | — | — | — |
| oil dimer dilinoleate | | | | | | | | | | |
| Butyrospermum Parkii | 2.0 | 2.0 | 2.0 | 2.0 | 2.0 | 2.0 | 2.0 | 2.0 | 2.0 | 2.0 |
| (Shea) butter | | | | | | | | | | |
| Phenoxyethanol | 0.5 | 0.5 | 0.5 | 0.5 | 0.5 | 0.5 | 0.5 | 0.5 | 0.5 | 0.5 |
| Caprylyl glycol | 0.2 | 0.2 | 0.2 | 0.2 | 0.2 | 0.2 | 0.2 | 02 | 0.2 | 0.2 |
| Tocopherol | 0.5 | 0.5 | 0.5 | 0.5 | 0.5 | 0.5 | 0.5 | 0.5 | 0.5 | 0.5 |
| Fragrance | 0.3 | 0.3 | 0.3 | 0.3 | 0.3 | 0.3 | 0.3 | 0.3 | 0.3 | 0.3 |

[Evaluation 1]

The removability, the spread ability during application and the feeling after rinsing-off of the compositions according to Examples 1-6 and Comparative Examples 1-4 were evaluated by 5 expert panelists who had applied proof mascara and foundation. The protocols for the evaluations are as follows.

(General Protocol)

2 g of each of the compositions according to Examples 1-6 and Comparative Examples 1-4 was applied on the whole face for 30 seconds, and then rinsed off by hot water. The removability, the spread ability during application and the feeling after rinsing-off were evaluated by the following criteria.

5: Excellent

4: Good

3: Somewhat Poor

2: Poor

1: Extremely Poor

The scores were averaged as follows.

∘∘: Excellent (5.0-4.0)

∘: Good (3.9-3.5)

Δ: Fair (3.4-3.0)

×: Somewhat Poor (2.9-2.0)

××: poor (1.9-1.0)

(1) Removability

The removability was determined by visually observing the residue of the applied proof mascara and foundation after rinsing-off the composition.

(2) Spread Ability During Application

The spread ability during application was determined based on ease of application without dripping down from fingers.

(3) Feeling After Rinsing-Off

The feeling after rinsing-off was determined sensorially 1 minute after towel dry.

[Evaluation 2]

The stability of the composition according to Examples 1-6 and Comparative Examples 1-4 was evaluated. In order to evaluate the stability of the composition, the sample of the composition was kept in an oven at 45° C. for 2 months, and then the sample was cooled for half a day to room temperature. The stability of the composition was determined by visually observing the surface of the sample as follows.

∘: no oil leakage

Δ: a little oil leakage

×: oil leakage

The results of the evaluations are shown in Table 2.

TABLE 2

| | | | | | | | Comparative | | | |
|---|---|---|---|---|---|---|---|---|---|---|
| | Examples | | | | | | Examples | | | |
| | 1 | 2 | 3 | 4 | 5 | 6 | 1 | 2 | 3 | 4 |
| Removability | ∘ | ∘ | ∘ | ∘ | ∘ | ∘ | ∘ | ∘ | ∘ | ∘ |
| Spread Ability | ∘ | ∘ | ∘∘ | Δ | ∘ | ∘∘ | ∘ | ∘ | ∘ | ∘∘ |
| during Application | | | | | | | | | | |
| Feeling after | ∘∘ | ∘ | ∘∘ | ∘ | Δ | Δ | x | ∘ | xx | ∘ |
| Rinsing-off | | | | | | | | | | |
| Stability | ∘ | ∘ | ∘ | ∘ | Δ | Δ | x | x | ∘ | x |

As shown in Table 2, all Examples show good or acceptable stability of the compositions. In contract, Comparative Examples 1, 2, and 4 lacking the wax or the oil gelling agent do not show good stability of the compositions. Comparative Example 3 lacking the nonionic surfactant shows good stability of the compositions, but shows a strong greasy feeling after rinsing-off. On the other hand, all Examples show good removability, excellent, good or acceptable spread ability during application and excellent, good or acceptable feeling after rinsing off. Examples 1-4 comprising Fispher-Tropsch wax show good stability of the compositions and excellent or good feeling after rinsing off.

1. A composition comprising:
(a) at least one liquid oil at 25° C.,
(b) at least one wax,
(c) at least one oil gelling agent derived from glutamic acid, and
(d) at least one nonionic surfactant.

2. The composition according to claim 1, wherein the (b) wax(es) is a synthetic wax.

3. The composition according to claim 2, wherein the (b) wax(es) comprises hydrocarbons containing odd and even numbers of carbon atoms, preferably Fisher-Tropsch wax.

4. The composition according to any one of claims 1 to 4, wherein the composition further comprises (e) at least one fatty alcohol.

5. The composition according to claim 4, wherein the amount of the (e) fatty alcohol(s) is from 0.5 to 15% by weight, preferably from 1 to 10% by weight, and more preferably from 2 to 8% by weight, relative to the total weight of the composition.

6. The composition according to any one of claims 1 to 5, wherein the amount of the (a) liquid oil(s) at 25° C. is from 50 to 90% by weight, preferably from 60 to 85% by weight, and more preferably from 65 to 80% by weight, relative to the total weight of the composition.

7. The composition according to any one of claims 1 to 6, wherein the amount of the (b) wax(es) is from 1 to 15% by weight, preferably from 3 to 10% by weight, and more preferably from 4 to 6% by weight, relative to the total weight of the composition.

8. The composition according to any one of claims 1 to 7, wherein the (c) oil gelling agent(s) is N-acyl glutamic acid dialkylamide.

9. The composition according to claim 8, wherein the N-acyl glutamic acid dialkylamide is N-acyl glutamic acid dibutylamide.

10. The composition according to any one of claims 1 to 9, wherein the amount of the (c) oil gelling agent(s) is from 0.01 to 5% by weight, preferably from 0.02 to 1% by weight, and more preferably from 0.03 to 0.5% by weight, relative to the total weight of the composition.

11. The composition according to any one of claims 1 to 10, wherein the HLB of the (d) nonionic surfactant(s) is from 8 to 13, preferably from 9 to 12, and more preferably from 10 to 11.

12. The composition according to any one of claims 1 to 11, wherein the amount of the (d) nonionic surfactant(s) is from 1 to 25% by weight, preferably from 5 to 20% by weight, and more preferably from 8 to 17% by weight, relative to the total weight of the composition.

13. The composition according to any one of claims 1 to 12, wherein the composition is solid at 25° C.

14. The composition according to any one of claims 1 to 13, wherein the composition is for a cosmetic use.

15. The composition according to claim 14, wherein the composition is for cleansing skin, nails, hair, eyelashes, eyebrows, eyes or mucous membranes.
